(19) United States
(12) Patent Application Publication
(10) Pub. No.: US 2026/0294353 A1
(43) Pub. Date: Oct. 1, 2026

(54) METHOD AND APPARATUS FOR PREDICTING BLOOD PRESSURE USING ARTIFICIAL INTELLIGENCE

(71) Applicant: IUCF-HYU (INDUSTRY-UNIVERSITY COOPERATION FOUNDATION HANYANG UNIVERSITY), Seoul (KR)

(72) Inventors: Joonhyuk CHANG, Seoul (KR); Jehyun KYUNG, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 19/478,470

(22) PCT Filed: Apr. 19, 2024
(86) PCT No.: PCT/KR2024/005360

§ 371 Date: Oct. 24, 2025

(30) Foreign Application Priority Data
Apr. 25, 2023 (KR) 10-2023-0054347

(51) Int. Cl. A61B 5/00; A61B 5/0205; A61B 5/021; A61B 5/024; A61B 5/318; G16H 50/30

(52) U.S. Cl. CPC A61B 5/7267; A61B 5/0205; A61B 5/021; A61B 5/02416; A61B 5/318; G16H 50/30

(57) ABSTRACT

In a method performed by an electronic device using artificial intelligence according to an embodiment of the present disclosure, the method comprises: receiving a photoplethysmogram (PPG) signal; and determining a blood pressure value in a pre-trained first artificial intelligence network using the PPG signal as input information, wherein the pre-trained first artificial intelligence network may be trained through a knowledge distillation method by a second artificial intelligence network that determines a blood pressure value based on demographic information using the PPG signal and an electrocardiogram (ECG) signal as input information.

### CROSS-REFERENCE TO RELATED APPLICATION

This application is a National Stage of International Application No. PCT/KR2024/005360, filed Apr. 19, 2024, claiming priority based on Korean Patent Application No. 10-2023-0054347 filed Apr. 25, 2023, the contents of which are incorporated herein by reference in their entireties.

### BACKGROUND

### 1. Field

The present disclosure relates to a method and an apparatus for performing blood pressure prediction using artificial intelligence. Specifically, the present disclosure relates to a method for performing blood pressure prediction by training an artificial intelligence with blood pressure-related information.

### 2. Description of the Related Art

The present disclosure relates to an apparatus and method for performing blood pressure prediction by analyzing heart-related information.

The mobile smart healthcare industry has been making great advancements for a target demographic that is aiming for a healthy life in an aging society. As a part of this, the demand for technology that can monitor one's own biometric indicators in devices such as smartwatches is increasing. Therefore, as a blood pressure prediction technology using artificial intelligence, a regular and accurate blood pressure prediction algorithm applicable to smartwatches may provide medical assistance to hypertensive patients. The present disclosure, which may provide medical assistance such as improving dietary habits and early diagnosis of hypertension while monitoring one's own healthy body condition even for the general public, is closely related to the healthcare industry.

Although there have been conventional blood pressure prediction technologies using artificial intelligence, the existing methods could derive accurate predicted blood pressure, but there was a problem in that they required a somewhat large amount of information, thus requiring additional sensors. Although various attempts have been made to supplement this, it was not easy to obtain accurate predicted blood pressure when the amount of information was reduced.

Therefore, there is a demand for a system that can predict blood pressure with only a small amount of information by monitoring a user's vital signs through a smart device as a blood pressure prediction algorithm using artificial intelligence.

### SUMMARY

The present disclosure is to provide a method and an apparatus for improving deep learning blood pressure prediction performance by training an artificial intelligence with only a small amount of information related to the heart.

The present disclosure is to provide a method for improving deep learning blood pressure prediction performance by training an artificial intelligence through focusing on necessary information in data.

A method according to an embodiment of the present disclosure, performed by an electronic device using artificial intelligence, comprises: receiving a photoplethysmogram (PPG) signal; and may include determining a blood pressure value in a pre-trained first artificial intelligence network using the PPG signal as input information. The pre-trained first artificial intelligence network may be trained through a knowledge distillation method by a second artificial intelligence network that determines a blood pressure value based on demographic information using the PPG signal and an electrocardiogram (ECG) signal as input information.

In an embodiment, the blood pressure value may include a systolic blood pressure value and a diastolic blood pressure value, and the demographic information may include at least one of height, age, and weight.

In an embodiment, the second artificial intelligence network may include an attention module, and the attention module may determine a weight for each signal based on the PPG signal and the ECG signal, and generate first data based on the weights.

In an embodiment, the second artificial intelligence network may generate second data based on the first data and the demographic information.

In an embodiment, the second artificial intelligence network may extract a joint embedding value based on the second data, and the step of determining the blood pressure value in the pre-trained first artificial intelligence network using the PPG signal as input information may include: extracting a PPG embedding value based on the PPG signal; and determining a loss based on the joint embedding value and the PPG embedding value.

In an embodiment, the first artificial intelligence network may be trained in a non-calibration manner.

In an embodiment, the first artificial intelligence network may be a student network, and the second artificial intelligence network may be a teacher network.

An electronic device according to an embodiment of the present disclosure comprises: a memory; a modem; and a processor connected to the modem and the memory, wherein the processor is configured to: receive a photoplethysmogram (PPG) signal, and determine a blood pressure value in a pre-trained first artificial intelligence network using the PPG signal as input information, and the pre-trained first artificial intelligence network may be trained through a knowledge distillation method by a second artificial intelligence network that determines a blood pressure value based on demographic information using the PPG signal and an electrocardiogram (ECG) signal as input information.

A program stored on a medium for performing a direction estimation method via an artificial intelligence algorithm executable by a processor, according to an embodiment of the present disclosure, performs the steps of: receiving a photoplethysmogram (PPG) signal; and determining a blood pressure value in a pre-trained first artificial intelligence network using the PPG signal as input information, and the pre-trained first artificial intelligence network may be trained through a knowledge distillation method by a second artificial intelligence network that determines a blood pressure value based on demographic information using the PPG signal and an electrocardiogram (ECG) signal as input information.

According to an embodiment of the present disclosure, it is possible to accurately predict blood pressure with only a small amount of information through artificial intelligence learning.

### BRIEF DESCRIPTION OF FIGURES

In order to more fully understand the drawings cited in the detailed description of the present disclosure, a brief description of each drawing is provided.

FIG. 1 is a conceptual diagram illustrating a basic principle of an artificial intelligence structure according to an embodiment of the present disclosure.

FIG. 2 is a diagram illustrating the structure of an artificial intelligence network according to an embodiment of the present disclosure.

FIG. 3 is a diagram illustrating the structure of an attention module according to an embodiment of the present disclosure.

FIG. 4 is a table showing the blood pressure prediction performance according to an artificial intelligence algorithm and various other algorithms according to an embodiment of the present disclosure.

FIG. 5 is a table showing the blood pressure prediction performance of an artificial intelligence algorithm according to an embodiment of the present disclosure and that in other studies.

FIG. 6 is a table comparing the performance of an artificial intelligence algorithm according to an embodiment of the present disclosure with medical standards.

FIG. 7 is a table comparing the performance of an artificial intelligence algorithm according to an embodiment of the present disclosure with another medical standard.

FIG. 8 is a block diagram of an electronic device for a blood pressure prediction system according to an embodiment of the present disclosure.

FIG. 9 is a flowchart for explaining a blood pressure prediction method using artificial intelligence according to an embodiment of the present disclosure.

### DETAILED DESCRIPTION OF EXEMPLARY EMBODIMENTS

The technical concept of the present disclosure may be subject to various modifications and may have various embodiments. Specific embodiments are illustrated in the drawings and described in detail herein. However, this is not intended to limit the technical concept of the present disclosure to specific forms, and it should be understood to include all modifications, equivalents, and alternatives within the scope of the technical concept of the present disclosure.

In describing the technical concept of the present disclosure, detailed descriptions of related known technologies may be omitted if they are deemed to obscure the gist of the present disclosure. In addition, numerical labels (e.g., first, second, etc.) used in the description are merely for distinguishing one component from another.

As used herein, when one component is described as being “connected to” or “coupled to” another component, it should be understood that the component may be directly connected or coupled to the other component, or may be indirectly connected or coupled through another component, unless otherwise stated.

The terms such as “~unit,” “~mechanism,” and “~er” described herein refer to a unit that processes at least one function or operation, and may be implemented with hardware such as a Processor, a Micro Processor, a Micro Controller, a Central Processing Unit (CPU), a Graphics Processing Unit (GPU), an Accelerate Processor Unit (APU), a Digital Signal Processor (DSP), an Application Specific Integrated Circuit (ASIC), a Field Programmable Gate Array (FPGA), or the like, software, or a combination of hardware and software.

And it is intended to clarify that the division of components in the present application is merely based on the main function performed by each component. That is, two or more components to be described below may be combined into one component, or one component may be provided by being divided into two or more parts according to more subdivided functions. Additionally, the classification of the components described herein is merely based on their respective main functions. Accordingly, two or more components may be combined into one, or a single component may be subdivided into two or more subcomponents by function. Each component may perform not only its main function but also part or all of the functions performed by other components. Conversely, part of the main function of a component may be dedicated to and performed by another component.

In describing embodiments of the present disclosure, detailed descriptions of related functions or configurations will be omitted when it is determined that they may unnecessarily obscure the gist of the present disclosure. The terms to be described later are terms defined in consideration of functions in the present disclosure, and may vary according to the intention or custom of the user or operator, and the like. Therefore, the definition should be made based on the entire content of the present specification.

For the same reason, some components in the attached drawings may be exaggerated, omitted, or schematically illustrated. In addition, the size of each component does not fully reflect the actual size. In each drawing, the same reference numerals are assigned to the same or corresponding components.

The advantages and features of the present disclosure and the method of achieving them will become clear by referring to the embodiments described in detail below with the attached drawings. However, the present disclosure is not limited to the embodiments disclosed below, but may be implemented in various different forms; rather, the embodiments are provided so that the description of the present disclosure is complete and to fully convey the scope of the invention to those skilled in the art to which the embodiments of the present disclosure pertain, and the scope to be claimed in the present disclosure is defined only by the scope of the claims.

At this time, it will be understood that each block of the flow chart illustrations, and combinations of the blocks in the flow chart illustrations, may be executed by computer program instructions. These computer program instructions may be mounted on a processor of a general purpose computer, a special purpose computer, or other programmable data processing equipment, so the instructions performed through the processor of the computer or other programmable data processing equipment create means for performing the functions described in the flowchart block(s). These computer program instructions may also be stored in a computer-usable or computer-readable memory that can direct a computer or other programmable data processing equipment to implement the functions in a particular manner, so the instructions stored in the computer-usable or computer-readable memory are capable of producing an article of manufacture including instruction means for performing the functions described in the flowchart block(s). The computer program instructions may also be loaded onto a computer or other programmable data processing equipment to cause a series of operational steps to be performed on the computer or other programmable data processing equipment to produce a computer implemented process such that the instructions that execute on the computer or other programmable data processing equipment provide steps for implementing the functions described in the flowchart block(s).

Also, each block may represent a module, segment, or portion of code that includes one or more executable instructions for implementing the specified logical function(s). It should also be noted that in some alternative implementations, the functions noted in the blocks may occur out of the order noted. For example, two blocks shown in succession may in fact be executed substantially concurrently or the blocks may sometimes be executed in the reverse order, depending upon the functionality involved.

The term ‘unit or part’ used in the present disclosure refers to a hardware component such as software or a Field-Programmable Gate Array (FPGA) or an Application Specific Integrated Circuit (ASIC), and the ‘unit’ may be configured to perform specific roles. However, a ‘unit’ is not limited to software or hardware. A ‘unit’ may be configured to reside on an addressable storage medium and configured to execute one or more processors. Thus, by way of example, a ‘unit’ may include components such as software components, object-oriented software components, class components, and task components, as well as processes, functions, attributes, procedures, subroutines, segments of program code, drivers, firmware, microcode, circuitry, data, databases, data structures, tables, arrays, and variables. The functionality provided by the components and ‘units’ may be combined into a smaller number of components and ‘units’ or further separated into additional components and ‘units’. Furthermore, the components and ‘units’ may be implemented to reproduce one or more CPUs within a device or a secure multimedia card. In an embodiment, a ‘unit’ may include one or more processors and/or devices. Hereinafter, embodiments according to the technical idea of the present disclosure will be described in detail in order.

Hereinafter, various embodiments according to the technical concept of the present disclosure will be described in detail.

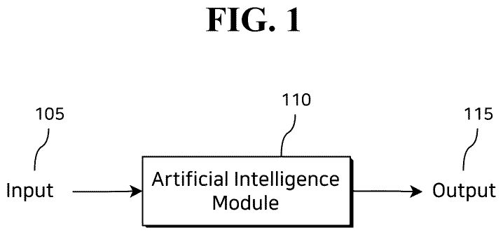

FIG. 1 is a conceptual diagram illustrating a basic principle of an artificial intelligence structure according to an embodiment of the present disclosure.

Referring to FIG. 1, the basic principle by which learning is performed in the artificial intelligence structure is illustrated.

The artificial intelligence technology refers to a technology for solving cognitive problems mainly associated with human intelligence, such as learning, problem solving, and recognition. The artificial intelligence may be trained through a machine learning method called Machine Learning (ML) and a deep learning method called Deep Learning (DL). Machine learning is mainly used for techniques used in pattern recognition and learning, and refers to an algorithm that predicts subsequent data based on learning from recorded data. The ML method refers to a technology that learns from data itself without being based on rules or patterns defined in advance. On the other hand, deep learning is a field of machine learning and has a difference in that the DL method processes data based on an Artificial Neural Network (ANN). Because deep learning uses an artificial neural network, the DL method may process more complex and sophisticated operations than machine learning. Types of algorithms for deep learning may include a Convolutional Neural Network (CNN), a deep neural network (DNN), an artificial neural network (ANN), and a Recurrent Neural Network (RNN).

Referring to FIG. 1, an artificial intelligence architecture may be represented by an artificial intelligence module 110. The artificial intelligence module 110 receives predetermined input data 105, performs training through a predetermined method in the module, and outputs output data 115 regarding a training result. According to an embodiment, the input data 105 may include predetermined data (e.g., an electrocardiogram (ECG), a photoplethysmogram (PPG), demographic features (height, age, weight), etc.), and an input sequence. The output data 115 may include an output sequence and a blood pressure value (a predicted blood pressure, a measured blood pressure, a systolic blood pressure (SBP), and a diastolic blood pressure (DBP)).

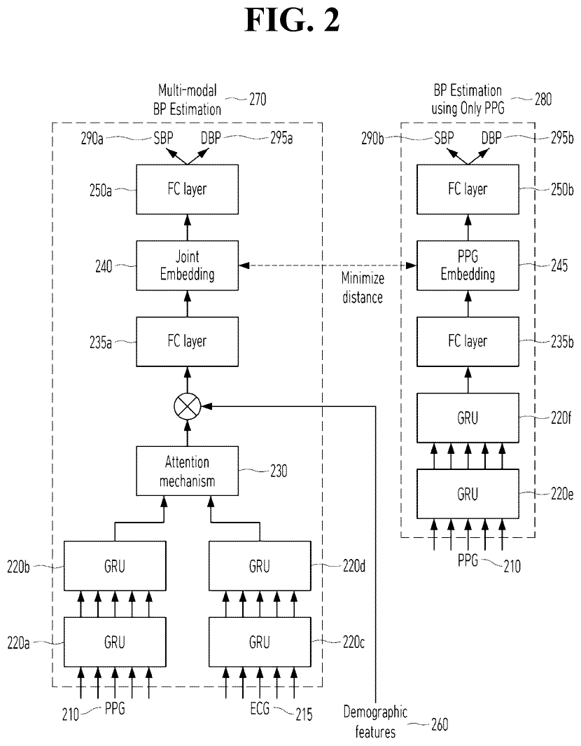

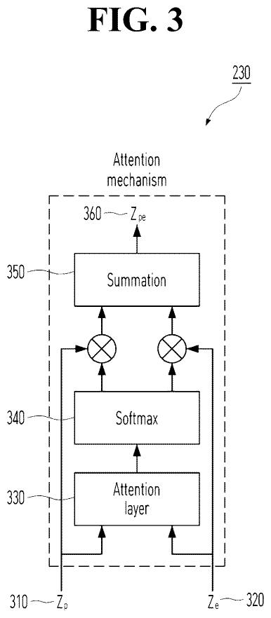

FIG. 2 is a diagram illustrating the structure of an artificial intelligence network according to an embodiment of the present disclosure. FIG. 3 is a diagram illustrating the structure of an attention module according to an embodiment of the present disclosure.

The artificial intelligence algorithm used in FIG. 2 may be one type of the artificial intelligence module 110 of FIG. 1.

Referring to FIG. 2, the structure of the artificial intelligence system may include blood pressure-related data and an artificial intelligence algorithm structure.

According to an embodiment, the blood pressure-related data may include a Photoplethysmogram (PPG) 210, an Electrocardiogram (ECG) 215, and demographic features (height, age, weight, etc.). The blood pressure-related data may be measured individually or together through at least one sensor included in a device.

According to an embodiment, the artificial intelligence algorithm structure may be divided into steps of data preprocessing, artificial intelligence training, and data output. Data preprocessing in the present disclosure may be performed in an attention module 230. The attention module 230 may include an attention layer 330, a softmax operation unit 340, and a summation operation unit 350.

The artificial intelligence algorithm of FIG. 2 is for ensuring that a model using only the PPG 210 as input information has the same performance as a multi-modal model 270. To derive this, a knowledge distillation technique called a teacher-student training technique may be applied. The knowledge distillation technique is a method of transferring knowledge from a large-scale model (teacher model) to a small model (student model). Accordingly, in FIG. 2, the artificial intelligence algorithm may be configured with a student network 280 and a teacher network 270. Here, the teacher network 270 may be the multi-modal model 270 which uses a plurality of inputs (e.g., the PPG, the ECG, etc.). Here, the student network 280 may be the model 280 (hereinafter, a PPG model) which uses only the PPG as an input.

The multi-modal model 270 of FIG. 2 may be configured with Gated Recurrent Unit (GRU) networks 220a to 220d, an attention module 230, a FC layer 235a, a joint embedding operation unit 240, and a FC layer 250a.

Referring to the multi-modal model 270 of FIG. 2, a Photoplethysmogram (PPG) signal (Xₚ) 210 and an Electrocardiogram (ECG) signal (Xₑ) 215, which are blood pressure-related data, may be respectively input to the GRU networks 220a to 220d in order to learn sequence information. A GRU network 220a to 220f may process sequence data as a variation of a Recurrent Neural Network (RNN). The GRU network has the advantage of having a simple structure but high computational efficiency. The GRU network may learn a long-term dependency of a sequence and may mitigate a gradient vanishing problem. Latent vectors (Zₚ and Zₑ) may be derived using Xₚ and Xₑ.

Referring to FIG. 3, the derived Zₚ 310 and Zₑ 320 are input to the attention module 230 and may be fused with the last frame output of the GRU networks 220a to 220d to derive a single latent vector (Zₚₑ) 360, where the Zₚₑ 360 may include the information of the input PPG and ECG signals. Here, Zₚₑ may calculate which ratio of fusion of Zₚ and Zₑ may further increase the blood pressure prediction performance. The attention module 230 uses Zₚ 310 and Zₑ 320 as inputs and may output attention weights (Wₚ and Wₑ) whose sum is 1. Then, Zₚₑ may be derived through Equation 1 below via a series of operation units (for example, an attention layer 330, a softmax operation unit 340, and a summation operation unit 350) based on Wₚ, Wₑ, and a weighted sum.

$$Zpe = {\sum }_{i} {Z}_{i} * \frac{exp ( w {Z}_{i} + b )}{{\sum }_{i} exp ( w {Z}_{i} + b )} , i ∈ [ PPG , ECG ] \qquad [ Equation 1 ]$$

Here, w represents a trainable weight and b represents a trainable bias.

Referring again to FIG. 2, the Zₚₑ 360 output from the attention module 230 may be connected to demographic information 260 (X_{d}). The demographic information 260 and the Zₚₑ 360 may be combined and input to a fully connected layer (FC layer) 235a. In the FC layer 235a, the Zₚₑ 360 and the X_{d} 260 are input to a jointly embedding operation unit, jointly embedded, and output E_{ped}. The jointly embedded E_{ped} is used as an input to a FC layer 250a, which is the last output layer of the multi-modal model 270, and may be converted into predicted values of SBP 290a and DBP 295a. The multi-modal model 270 may be optimized with a Mean Squared Error (MSE) loss function, which is often used in a blood pressure prediction task, using the predicted blood pressure value and the measured blood pressure value. The equation for calculating the MSE loss function using SBP 290a and DBP 295a is as follows.

$${ℒ}_{MSE} = \frac{1}{N} {\sum }_{j} {\sum }_{i = 1}^{N} {( {y}_{i} - {\hat{y}}_{i} )}^{2} , j ∈ [ SBP , DBP ] \qquad [ Equation 2 ]$$

Here, N may represent a mini batch size.

In order to apply the knowledge distillation technique, the multi-modal model 270 may become the teacher network and the PPG model 280 may become the student network. The components of the PPG model 280 may be similar to those of the multi-modal model 270.

The PPG model 280 of FIG. 2 may be configured with GRU networks 220e and 220f, a FC layer 235b, a PPG embedding operation unit 245, and a FC layer 250b.

Referring to the PPG model 280 of FIG. 2, only the PPG signal 210 may be used as an input signal, excluding the ECG signal and the demographic information. The input PPG signal 210 Xₚ is input to the GRU networks 220e, 220f, and sequence data may be processed. The PPG model 280 does not include the attention module 230, unlike the multi-modal model 270, so the PPG model 280 is immediately input to the FC layer 235b and input to the PPG embedding operation unit 245, and may output Eₚ, which is the embedding of the PPG signal. The knowledge distillation technique may be applied so that the distance between Eₚ and the E_{ped} of the multi-modal model 270 is minimized. The distance between Eₚ and E_{ped} is called the L-2 distance, and this distance may be calculated with the following equation.

$${ℒ}_{dist} = ‖ {E}_{ped} - {E}_{p} ‖ \qquad [ Equation 3 ]$$

After that, the FC layer 250b may output the predicted values of SBP 290b and DBP 295b. The final loss function for training the PPG model 280 may be calculated as in the following equation.

$${ℒ}_{total} = {αℒ}_{MSE} + {βℒ}_{dist} \qquad [ Equation 4 ]$$

Here, α and β may represent hyperparameters. α and β may be set to values greater than or equal to 0 and less than or equal to 1. For example, α may be set to 1, and β may be set to 0.5. The PPG module 280 may be trained based on the final loss function.

If the trained network is applied to a smart device (e.g., a smart watch, a smart phone), only a sensor that acquires only the PPG signal needs to be applied to the corresponding device, thereby providing a cost reduction effect and high-accuracy blood pressure prediction performance.

In addition, the multi-modal model 270 and the PPG model 280 of FIG. 2 may be trained in a non-calibration manner. The calibration method is a method of compensating the blood pressure output from the blood pressure prediction system based on existing reference blood pressure or information on an input signal. However, the calibration method may incur additional cost and time since the reference blood pressure must be measured separately. However, the artificial intelligence algorithm of the present disclosure is trained in a non-calibration manner and may be applied to the blood pressure prediction system with only input information to obtain a predicted blood pressure value.

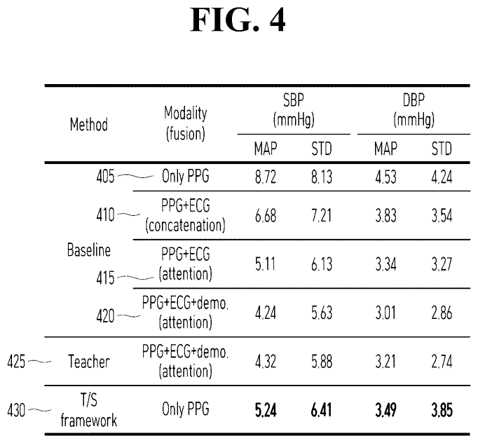

FIG. 4 is a table showing the blood pressure prediction performance according to an artificial intelligence algorithm and various other algorithms according to an embodiment of the present disclosure.

The experiment of FIG. 4 may indicate the prediction accuracy of SBP and DBP when various algorithms are trained based on PPG, ECG, and demographic information. In particular, it is illustrated through MAP (maximum a posteriori) and STD (standard deviation), and it can be understood that a lower value in both indicates higher performance.

Referring to FIG. 4, a model 405 that predicts blood pressure by learning using only PPG as an existing model, a model 410 that predicts blood pressure by learning using PPG and ECG using a concatenation technique, a model 415 that predicts blood pressure by learning using PPG and ECG through an attention module, a model 420 that predicts blood pressure by learning using PPG and ECG with demographic information added through an attention module, a multi-modal model 425 (e.g., the multi-modal model 270 of FIG. 2), and a PPG model 430 (e.g., the PPG model 280 of FIG. 2) trained through the knowledge distillation technique were utilized in the experiment. The multi-modal model 425 and the model 420 that predicts blood pressure by learning using PPG and ECG with demographic information added through the attention module may be similar models.

The data set used in the present disclosure and the present experiment is the MIMIC III data set, which is a public data set. The MIMIC III data set is a large-scale data set of electronic health records (EHR) publicly available for patients hospitalized in intensive care units.

Referring to the results of FIG. 4, the accuracy was higher when the PPG and ECG signals were used together than when only the PPG signal was used. In addition, it can be confirmed that the accuracy is higher when the attention module is used than when the concatenation technique is used. The attention module can improve accuracy rather than simply connecting signals because it fuses features of PPG and ECG signals at an appropriate ratio. In the case of the multi-modal model 425, there was a significant performance improvement, and in the case of the PPG model 430 when the knowledge distillation technique was applied, although it did not show performance as high as that of the multi-modal model 425, it was confirmed that it showed performance comparable to the model 415 using the attention module for PPG and ECG even though it only used the PPG signal. However, it can be confirmed that the performance is excellent when based on MAP, but it can be confirmed that the improvement is relatively less when based on STD.

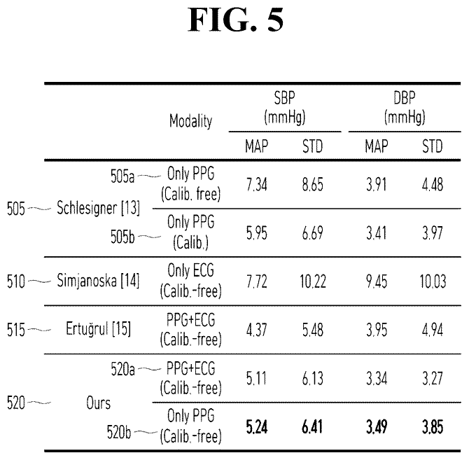

FIG. 5 is a table showing the blood pressure prediction performance of an artificial intelligence algorithm according to an embodiment of the present disclosure and that in other studies.

FIG. 5 compares the performance of models using or not using the calibration method with the performance of a model not using the calibration method according to an embodiment of the present disclosure.

The Schlesinger 505 experiment in FIG. 5 compares the performance of a model 505b that used the calibration method and a model 505a that did not use the calibration method when training the artificial intelligence model using only PPG. It can be confirmed that the model 505b that used the calibration method has better performance than the model 505a that did not use the calibration method. Similarly, in the Simjanoska 510 and Ertugrul 515 experiments, when the calibration method was not used, the performance changed based on what was used as input data. It can be confirmed that Ertugrul 515, which used both PPG and ECG as input data, shows good performance. The model 520 according to an embodiment of the present disclosure did not use the calibration method, and although the performance is lower than that of the model 520a that used both PPG and ECG as input data, the model 520b that used only PPG as input (e.g., the PPG model 280 of FIG. 2) was confirmed to have better performance than the model 505a that did not use calibration in the Schlesinger study.

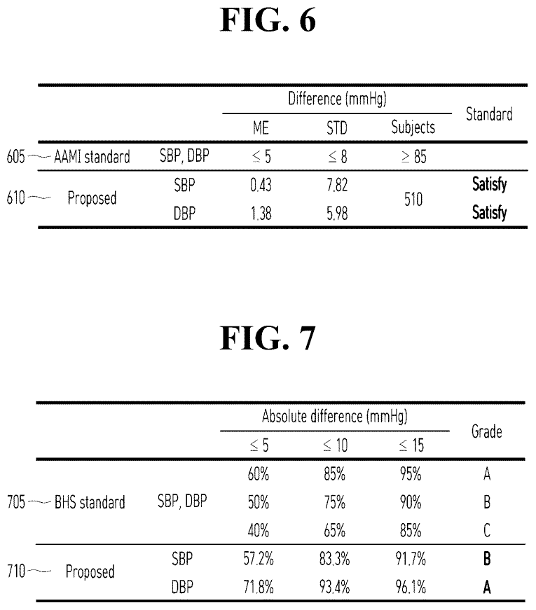

FIG. 6 is a table comparing the performance of an artificial intelligence algorithm according to an embodiment of the present disclosure with medical standards.

FIG. 6 shows the standards presented by AAMI (Association for the Advancement of Medical Instrumentation), a non-profit organization in the United States, and the blood pressure values predicted through the artificial intelligence algorithm (e.g., the PPG model 280) according to an embodiment of the present disclosure.

The AAMI standard 605 specifies that for SBP and DBP, ME should be 5 or less, STD should be 8 or less, and the subject count should be 85 or more.

Referring to FIG. 6, the proposed artificial intelligence 610 shows ME of 0.43, STD of 7.82, and a subject count of 510 for SBP, and ME of 1.38, STD of 5.98, and a subject count of 510 for DBP, which indicates that the proposed artificial intelligence satisfies the standard criteria.

FIG. 7 is a table comparing the performance of an artificial intelligence algorithm according to an embodiment of the present disclosure with another medical standard.

FIG. 7 shows the criteria evaluated by the BHS (British Hypertension Society), a blood pressure-related organization in the UK, and the blood pressure values predicted through the artificial intelligence algorithm (e.g., the PPG model 280) according to an embodiment of the present disclosure.

The BHS standard 705 gives an A grade if the absolute difference for SBP and DBP is 60% or more for 5 or less, 85% or more for 10 or less, and 95% or more for 15 or less. Furthermore, the BHS standard gives a B grade if the absolute difference is 5 or less for 50% or more, 10 or less for 75% or more, and 15 or less for 90% or more. The BHS standard also gives a C grade if the absolute difference is 5 or less for 40% or more, 10 or less for 65% or more, and 15 or less for 85% or more.

Referring to FIG. 7, for SBP, the proposed artificial intelligence 710 shows an absolute difference of 5 or less for 57.2%, 10 or less for 83.3%, and 15 or less for 91.7%, which corresponds to a B grade. For DBP, the proposed artificial intelligence 710 shows an absolute difference of 5 or less for 71.8%, 10 or less for 93.4%, and 15 or less for 96.1%, which corresponds to an A grade.

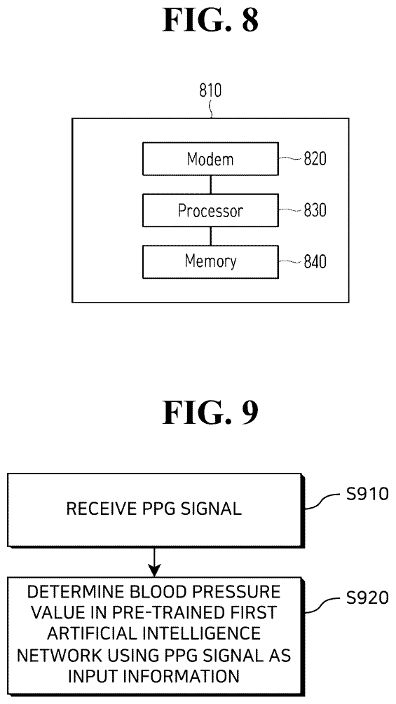

FIG. 8 is a block diagram of an electronic device for a blood pressure prediction system according to an embodiment of the present disclosure.

Referring to FIG. 8, an electronic device 810 may include a modem 820, a memory 840, and a processor 830.

The modem 820 may be a communication modem that is electrically connected to other electronic devices to enable mutual communication. Specifically, the modem 820 may receive data input and transmit the data input to the processor 830, and the processor 830 may store the input data value in the memory 840. In addition, the modem 820 may transmit the data value output by the artificial intelligence algorithm trained in the system to another electronic device.

The memory 840 is a component in which various information and program instructions for the operation of the electronic device 810 are stored, and may be a storage device such as a Hard Disk or a Solid State Drive (SSD). Specifically, the memory 840 may store one or more data input values input from the modem 820 under the control of the processor 830. In addition, the memory 840 may store program instructions, such as an artificial intelligence algorithm for blood pressure prediction, executable by the processor 830. Furthermore, the memory 840 may store a program (or program instructions), such as the artificial intelligence algorithm (e.g., the PPG model 280 of FIG. 2) trained through knowledge distillation described in the present disclosure.

The processor 830 is configured with at least one processor, and may train an artificial intelligence algorithm related to voice event detection using the data and program instructions stored in the memory 840 and calculate data using the trained algorithm. The processor 830 may control and calculate all the artificial intelligence algorithms described in FIG. 1 to FIG. 7. The processor 830 may perform the operation for the method to be described later in FIG. 9.

FIG. 9 is a flowchart for explaining a blood pressure prediction method using artificial intelligence according to an embodiment of the present disclosure.

Hereinafter, with reference to FIG. 9, the training operation of the artificial intelligence algorithm and the blood pressure prediction method of the electronic device described with reference to FIG. 1 to FIG. 8 are summarized and described. Each operation may not be an operation that must be included in a series of processes, and only a part of the operations may be configured to operate depending on the situation.

In step S910, a PPG signal may be received. The PPG signal may be received from a PPG sensor included in a device.

In step S920, a blood pressure value may be determined in a pre-trained first artificial intelligence network using the PPG signal as input information. The pre-trained first artificial intelligence network may be trained through a knowledge distillation technique by a second artificial intelligence network which determines the blood pressure value based on demographic information, using the PPG signal and an ECG signal as input information. The blood pressure value may include a systolic blood pressure value and a diastolic blood pressure value, and the demographic information may include at least one of height, age, and weight. The second artificial intelligence network includes an attention module, and the attention module may determine a weight for each signal based on the PPG signal and the ECG signal, and may generate first data based on the weight. The second artificial intelligence network may generate second data by further adding the first data and the demographic information. The second artificial intelligence network extracts a joint embedding value based on the second data, and the step of determining the blood pressure value in the pre-trained first artificial intelligence network using the PPG signal as input information may include: a step of extracting a PPG embedding value based on the PPG signal; and a step of determining a loss based on the joint embedding value and the PPG embedding value. The first artificial intelligence network may be trained in a non-calibration manner. The first artificial intelligence network may be a student network, and the second artificial intelligence network may be a teacher network.

The technical idea of the present disclosure has been described in detail with various embodiments as above, but the technical idea of the present disclosure is not limited to the above embodiments, and various modifications and changes are possible by those skilled in the art within the scope of the technical idea of the present disclosure.

What is claimed is:

1. A method performed by an electronic device using artificial intelligence, the method comprising:
receiving a photoplethysmogram (PPG) signal; and
determining a blood pressure value in a pre-trained first artificial intelligence network using the PPG signal as input information,
wherein the pre-trained first artificial intelligence network is trained through a knowledge distillation method by a second artificial intelligence network that determines a blood pressure value based on demographic information using the PPG signal and an electrocardiogram (ECG) signal as input information.

2. The method of claim 1, wherein the blood pressure value includes a systolic blood pressure value and a diastolic blood pressure value, and the demographic information includes at least one of height, age, and weight.

3. The method of claim 1, wherein the second artificial intelligence network includes an attention module, and
the attention module is configured to determine a weight for each signal based on the PPG signal and the ECG signal, and generate first data based on the weights.

4. The method of claim 3, wherein the second artificial intelligence network generates second data based on the first data and the demographic information.

5. The method of claim 4, wherein the second artificial intelligence network extracts a joint embedding value based on the second data, and
the determining of the blood pressure value in the pre-trained first artificial intelligence network using the PPG signal as input information comprises:
extracting a PPG embedding value based on the PPG signal; and
determining a loss based on the joint embedding value and the PPG embedding value.

6. The method of claim 1, wherein the first artificial intelligence network is trained in a non-calibration manner.

7. The method of claim 1, wherein the first artificial intelligence network is a student network, and the second artificial intelligence network is a teacher network.

8. An electronic device, comprising:
a memory;
a modem; and
a processor connected to the modem and the memory,
wherein the processor is configured to:
receive a photoplethysmogram (PPG) signal, and
determine a blood pressure value in a pre-trained first artificial intelligence network using the PPG signal as input information,
wherein the pre-trained first artificial intelligence network is trained through a knowledge distillation method by a second artificial intelligence network that determines a blood pressure value based on demographic information using the PPG signal and an electrocardiogram (ECG) signal as input information.

9. The electronic device of claim 8, wherein the blood pressure value includes a systolic blood pressure value and a diastolic blood pressure value, and
the demographic information includes at least one of height, age, and weight.

10. The electronic device of claim 8, wherein the second artificial intelligence network includes an attention module, and
the attention module is configured to determine a weight for each signal based on the PPG signal and the ECG signal, and generate first data based on the weights.

11. The electronic device of claim 10, wherein the second artificial intelligence network is configured to generate second data based on the first data and the demographic information.

12. The electronic device of claim 11, wherein the second artificial intelligence network is configured to extract a joint embedding value based on the second data, and
the processor is further configured to:
extract a PPG embedding value based on the PPG signal, determine a loss based on the joint embedding value and the PPG embedding value, and perform training based on the loss.

13. The electronic device of claim 8, wherein the first artificial intelligence network is trained in a non-calibration manner.

14. The electronic device of claim 8, wherein the first artificial intelligence network is a student network, and the second artificial intelligence network is a teacher network.

15. A program stored on a medium for performing a direction estimation method via an artificial intelligence algorithm executable by a processor, the program causing the processor to perform operations comprising:
receiving a photoplethysmogram (PPG) signal; and
determining a blood pressure value in a pre-trained first artificial intelligence network using the PPG signal as input information,
wherein the pre-trained first artificial intelligence network is trained through a knowledge distillation method by a second artificial intelligence network that determines a blood pressure value based on demographic information using the PPG signal and an electrocardiogram (ECG) signal as input information.
